(19) United States
(12) Patent Application Publication
(10) Pub. No.: US 2010/0262188 A1
(43) Pub. Date: Oct. 14, 2010

(54) Photodynamic Bone Stabilization Systems and Methods for Treating Spine Conditions

(75) Inventors: Robert A. Rabiner, Tiverton, RI (US); Dennis P. Colleran, North Attleboro, MA (US); Justin G. Dye, Mansfield, MA (US)

Correspondence Address: GREENBERG TRAURIG, LLP, ONE INTERNATIONAL PLACE, 20th FL, ATTN: PATENT ADMINISTRATOR, BOSTON, MA, 02110, US

(73) Assignee: IlluminOss Medical, Inc.

(21) Appl. No.: 12/756,014

(22) Filed: Apr. 7, 2010

Related U.S. Application Data
(60) Provisional application No. 61/167,299, filed on Apr. 7, 2009.

(51) Int. Cl. A61B 17/70

(52) U.S. Cl. 606/249

(57) ABSTRACT

In an embodiment, an interspinous process spacer system includes a light-conducting fiber configured to transmit light energy; a liquid light-curable material; and a catheter having an elongated shaft with a proximal end adapter, a distal end releasably engaging an expandable interspinous process spacer device, and a longitudinal axis therebetween, wherein an inner void of the catheter is sufficiently designed for passage of the liquid light-curable material to the interspinous process spacer device, wherein an inner lumen of the catheter is sufficiently designed for passage of the light-conducting fiber to the interspinous process spacer device, wherein the interspinous process spacer device includes a circumferential groove, wherein the interspinous process spacer device is sufficiently designed to inflate and deflate as the liquid light-curable material is added, and wherein the interspinous process spacer device, when positioned between two spinous processes and inflated, is configured to engage the spinous processes at the groove.

### RELATED APPLICATIONS

This application claims the benefit of and priority to U.S. Provisional Application Ser. No. 61/167,299, filed Apr. 7, 2009, the entirety of this application is hereby incorporated herein by reference.

### FIELD

The presently disclosed embodiments relate to systems and methods for treating the spine, and more particularly to photodynamic bone stabilization systems and methods for treating spine conditions, for example, spinal stenosis and degenerative disc disease.

### BACKGROUND

Degenerative disc disease (DDD) of the spine is one of the most common causes of lower back pain. The discs and the facet joints are considered the motion segments of the vertebral columns; the discs also act as shock absorbers between the vertebral bodies. Two prevalent causes of degenerative disc disease are increased thinning of the disc due to age, and thinning due to injury, for instance when the vertebral endplate tears from its connection to the intervertebral disc. Disc replacement goals include eliminating pain, sustaining range of motion, protecting adjacent spine segments, reducing morbidity and restoration of disc height.

Spinal stenosis is the narrowing of one or more areas in the spinal canal, frequently in the upper or lower back. This narrowing can put pressure on the spinal cord or on the nerves that branch out from the compressed areas, causing numbness and pain. Various different surgery options are available to a patient having spinal stenosis, including, but not limited to, spinal fusion surgery, spinal laminectomy surgery, and interspinous process spacer surgery.

### SUMMARY

Photodynamic bone stabilization systems and methods for treating spine conditions are disclosed herein.

According to aspects illustrated herein, there is disclosed an interspinous process spacer system that includes a light-conducting fiber configured to transmit light energy; a liquid light-curable material; and a catheter having an elongated shaft with a proximal end adapter, a distal end releasably engaging an expandable interspinous process spacer device, and a longitudinal axis therebetween, wherein an inner void of the catheter is sufficiently designed for passage of the liquid light-curable material to the expandable interspinous process spacer device, wherein an inner lumen of the catheter is sufficiently designed for passage of the light-conducting fiber to the expandable interspinous process spacer device, wherein the expandable interspinous process spacer device includes a circumferential groove, wherein the expandable interspinous process spacer device is sufficiently designed to inflate and deflate as the liquid light-curable material is added, and wherein the expandable interspinous process spacer device, when positioned between two spinous processes and inflated, is configured to engage the spinous processes at the groove.

According to aspects illustrated herein, there is disclosed a method that includes providing an interspinous process spacer system comprising: a light-conducting fiber configured to transmit light energy; a liquid light-curable material; and a catheter having an elongated shaft with a proximal end adapter, a distal end releasably engaging an expandable interspinous process spacer device, and a longitudinal axis therebetween, wherein an inner void of the catheter is sufficiently designed for passage of the liquid light-curable material to the expandable interspinous process spacer device, wherein an inner lumen of the catheter is sufficiently designed for passage of the light-conducting fiber to the expandable interspinous process spacer device, wherein the expandable interspinous process spacer device includes a circumferential groove, and wherein the expandable interspinous process spacer device is sufficiently designed to inflate and deflate as the liquid light-curable material is added; positioning the expandable interspinous process spacer device between two spinous processes; infusing the liquid light-curable material into the expandable interspinous process spacer device to inflate the expandable interspinous process spacer device, wherein the groove of the expandable interspinous process spacer device engages the spinous processes; inserting the light-conducting fiber into the inner lumen of the catheter so that the light-conducting fiber resides in the expandable interspinous process spacer device; activating the light-conducting fiber to transmit light energy to the expandable interspinous process spacer device to initiate polymerization of the liquid light-curable material within the expandable interspinous process spacer device; and completing the polymerization of the liquid light-curable material to harden the expandable interspinous process spacer device.

According to aspects illustrated herein, there is disclosed a method that includes providing a system comprising: a light-conducting fiber configured to transmit light energy; a liquid light-curable material; and a catheter having an elongated shaft with a proximal end adapter, a distal end releasably engaging an expandable interbody device, and a longitudinal axis therebetween, wherein an inner void of the catheter is sufficiently designed for passage of the liquid light-curable material to the expandable interbody device, wherein an inner lumen of the catheter is sufficiently designed for passage of the light-conducting fiber to the expandable interbody device, and wherein the expandable interbody device is sufficiently designed to inflate and deflate as the liquid light-curable material is added; removing at least a portion of a damaged intervertebral disc, the damaged intervertebral disc positioned between an upper vertebral body and a lower vertebral body; inserting the expandable interbody device between the upper vertebral body and the lower vertebral body in place of the damaged intervertebral disc; infusing the liquid light-curable material into the expandable interbody device to inflate the expandable interbody device; inserting the light-conducting fiber into the inner lumen of the catheter so that the light-conducting fiber resides in the expandable interbody device; activating the light-conducting fiber to transmit light energy to the expandable interbody device to initiate polymerization of the liquid light-curable material within the expandable interbody device; and completing the polymerization of the liquid light-curable material to harden the expandable interbody device, wherein at least a portion of an outer surface of the hardened expandable interbody device engages the upper vertebral body and the lower vertebral body.

According to aspects illustrated herein, there is disclosed a method that includes providing a system comprising: a light-conducting fiber configured to transmit light energy; a liquid light-curable material; and a catheter having an elongated shaft with a proximal end adapter, a distal end releasably engaging an expandable spinal fusion device, and a longitudinal axis therebetween, wherein an inner void of the catheter is sufficiently designed for passage of the liquid light-curable material to the expandable spinal fusion device, wherein an inner lumen of the catheter is sufficiently designed for passage of the light-conducting fiber to the expandable spinal fusion device, and wherein the expandable spinal fusion device is sufficiently designed to inflate and deflate as the liquid light-curable material is added; placing pedicle screws at consecutive spine segments, each of the pedicle screws having openings; inserting the expandable spinal fusion device into the openings of the pedicle screws to connect the pedicle screws together; infusing the liquid light-curable material into the expandable spinal fusion device to inflate the expandable spinal fusion device; inserting the light-conducting fiber into the inner lumen of the catheter so that the light-conducting fiber resides in the expandable spinal fusion device; activating the light-conducting fiber to transmit light energy to the expandable spinal fusion device to initiate polymerization of the liquid light-curable material within the expandable spinal fusion device; and completing the polymerization of the liquid light-curable material to harden the expandable spinal fusion device, wherein the hardened expandable spinal fusion device is sufficiently designed to fixate the spine segment.

### BRIEF DESCRIPTION OF THE DRAWINGS

The presently disclosed embodiments will be further explained with reference to the attached drawings, wherein like structures are referred to by like numerals throughout the several views. The drawings shown are not necessarily to scale, with emphasis instead generally being placed upon illustrating the principles of the presently disclosed embodiments.

FIG. 1 shows a proximal end of an embodiment of a flexible insertion catheter of the present disclosure. A spinal device of the present disclosure is releasably mounted at a distal end of the flexible insertion catheter.

FIG. 2 shows an isometric view of an embodiment of a spinal device or “interbody device” of the present disclosure.

FIG. 3 shows a perspective view of the interbody device of FIG. 2 positioned between two vertebrae of a spinal column for the treatment of, for example, degenerative disc disease (DDD). The interbody device is being positioned using the flexible insertion catheter of FIG. 1.

FIG. 4 shows a top-down plan view taken along line A-A of FIG. 3.

FIG. 5 shows a side sectional view taken along line B-B of FIG. 4.

FIG. 6 shows a perspective view of an embodiment of a spinal device or “spinal fusion device” of the present disclosure being positioned between two pedicles of a spinal column for the treatment of, for example, spinal stenosis. The spinal fusion device is positioned using the flexible insertion catheter of FIG. 1. In FIG. 6, the spinal fusion device is positioned between two pedicle screws engaging the pedicles.

FIG. 7 shows a top-down plan view of an embodiment of a spinal device or “spinal fusion device” of the present disclosure being positioned between two pedicles of a spinal column for the treatment of, for example, spinal stenosis. The spinal fusion device is positioned using the flexible insertion catheter of FIG. 1. In FIG. 7, the spinal fusion device is positioned between two pedicle screws engaging the pedicles.

FIG. 8 shows a side perspective view of an embodiment of a spinal device or “interspinous process spacer device” of the present disclosure between two spinous processes of a spinal column for the treatment of, for example, spinal stenosis. The interspinous process spacer device is positioned using the flexible insertion catheter of FIG. 1.

FIG. 9 shows a top-down plan view showing a distal end of the flexible insertion catheter of FIG. 1 positioning the interspinous process spacer device of FIG. 8 between the two spinous processes.

FIG. 10 shows a back perspective view of the interspinous process spacer device of FIG. 8 positioned between two spinous processes during expansion of the spacer.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

The embodiments disclosed herein relate to minimally invasive orthopedic procedures and more particularly to photodynamic bone stabilization systems for treating spine conditions. In an embodiment, a photodynamic bone stabilization system of the present disclosure includes a thin-walled, non-compliant, interbody device releasably mounted on a small diameter, flexible insertion catheter. The interbody device can be used in a procedure for treating degenerative disc disease (DDD). In an embodiment, a photodynamic bone stabilization system of the present disclosure includes a thin-walled, non-compliant, spinal fusion device releasably mounted on a small diameter, flexible insertion catheter. The spinal fusion device can be used in a procedure for treating spinal stenosis. In an embodiment, a photodynamic bone stabilization system of the present disclosure includes a thin-walled, non-compliant, interspinous process spacer device releasably mounted on a small diameter, flexible insertion catheter. The interspinous process spacer device can be used in a procedure for treating spinal stenosis. Generally, the interbody devices, spinal fusion devices, and interspinous process spacer devices of the present disclosure are referred to herein as “spinal devices”.

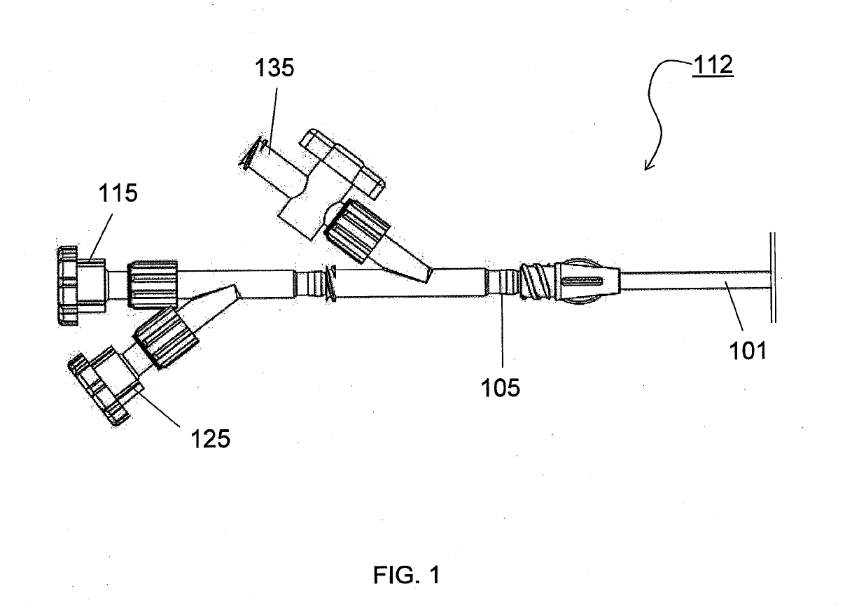

FIG. 1 shows an embodiment of a proximal end 112 of a flexible insertion catheter 101 of a photodynamic bone stabilization system of the present disclosure for treating spine conditions. The photodynamic bone stabilization system includes a thin-walled, non-compliant, expandable spinal device (not visible in FIG. 1) releasably mounted at a distal end of the flexible insertion catheter 101. In an embodiment, the flexible insertion catheter 101 includes one or more radiopaque markers or bands positioned at various locations. The one or more radiopaque bands, using radiopaque materials such as barium sulfate, tantalum, or other materials known to increase radiopacity, allows a medical professional to view the insertion catheter 101 using fluoroscopy techniques. A proximal end adapter 105 includes at least one arm and at least one adapter which can be utilized for the infusion and withdrawal of fluids or as conduits for the introduction of devices (e.g., a light-conducting fiber). In an embodiment, an adapter is a Luer lock. In an embodiment, an adapter is a Tuohy-Borst connector. In an embodiment, an adapter is a multi-functional adapter. FIG. 1 shows a side view of a three arm proximal end fitting having three adapters 115, 125, and 135. Adapter 115 can accept, for example, a light-conducting fiber. Adapter 125 can accept, for example, air or fluid. In an embodiment, adapter 125 can accept, for example, a cooling medium. In an embodiment, adapter 125 can accept, for example, pressurizing medium. Adapter 135 can accept, for example, a syringe housing a liquid light-curable material (also referred to herein as a “photodynamic material” or a “light-sensitive liquid monomer”). In an embodiment, the liquid light-curable material is a liquid monomer comprising an initiator, wherein the initiator is activated when the light-conducting fiber transmits light energy. In an embodiment, the viscosity of the liquid light-curable material is about 1000 cP or less. In an embodiment, the liquid light-curable material has a viscosity ranging from about 650 cP to about 450 cP. Low viscosity allows filling of the spinal device through a very small delivery system.

In an embodiment, a syringe housing light-sensitive liquid is attached to the adapter 135 at the proximal end 112 of the insertion catheter 101, and during use of the photodynamic bone stabilization system, the syringe plunger is pushed, allowing the syringe to expel the liquid light-curable material into an inner void 110 (not visible in FIG. 1) of the photodynamic bone stabilization system. As the liquid light-curable material is expelled through the inner void, the liquid light-curable material reaches the spinal device to move the spinal device from a deflated state to an inflated state. The liquid light-curable material can be aspirated and reinfused as necessary, allowing for adjustments to the spinal device prior to curing of the liquid light-curable material, wherein curing of the liquid light-curable material hardens the expandable spinal device in a desired position to form stabilization. The liquid light-curable material can be aspirated and reinfused as necessary, allowing for adjustments to the expandable spinal device. These properties allow a user to achieve a desired result prior to activating a light source and converting the liquid monomer into a hard polymer.

In an embodiment, a light-conducting fiber communicating light from a light source is introduced into adapter 115 at the proximal end 112 of the insertion catheter 101 to pass the light-conducting fiber within an inner lumen 120 (not visible in FIG. 1) of the photodynamic bone stabilization system up into the expandable spinal device. In an embodiment, the insertion catheter 101 is sufficiently designed so that the inner lumen of the insertion catheter 101 is separated from the inner void of the insertion catheter 101 so that light-conducting fiber and the liquid light-curable material do not directly contact one another down the length of the insertion catheter 101 shaft. The liquid light-curable material remains a liquid monomer until activated by the light-conducting fiber (cures on demand). In an embodiment, radiant energy from the light source is absorbed and converted to chemical energy to quickly polymerize the monomer. This cure affixes the expandable spinal device in an expanded shape. A cure may refer to any chemical, physical, and/or mechanical transformation that allows a composition to progress from a form (e.g., flowable form) that allows the composition to be delivered through the inner void in the flexible insertion catheter 101, into a more permanent (e.g., cured) form for final use in situ. For example, “curable” may refer to uncured composition, having the potential to be cured in situ (as by catalysis or the application of a suitable energy source), as well as to a composition in the process of curing (e.g., a composition formed at the time of delivery by the concurrent mixing of a plurality of composition components).

In an embodiment, the light-conducting fiber is an optical fiber. Optical fibers may be used in accordance with the present disclosure to communicate light from the light source to the remote location. Optical fibers use a construction of concentric layers for optical and mechanical advantages. The most basic function of a fiber is to guide light, i.e., to keep light concentrated over longer propagation distances—despite the natural tendency of light beams to diverge, and possibly even under conditions of strong bending. In the simple case of a step-index fiber, this guidance is achieved by creating a region with increased refractive index around the fiber axis, called the fiber core, which is surrounded by the cladding. The cladding is usually protected with at least a polymer coating. Light is kept in the “core” of the optical fiber by total internal reflection. Cladding keeps light traveling down the length of the fiber to a destination. In some instances, it is desirable to conduct electromagnetic waves along a single guide and extract light along a given length of the guide's distal end rather than only at the guide's terminating face. In some embodiments of the present disclosure, at least a portion of a length of an optical fiber is modified, e.g., by removing the cladding, in order to alter the direction, propagation, amount, intensity, angle of incidence, uniformity and/or distribution of light.

The optical fiber can be made from any material, such as glass, silicon, silica glass, quartz, sapphire, plastic, combinations of materials, or any other material, and may have any diameter, as not all embodiments of the present disclosure are intended to be limited in this respect. In an embodiment, the optical fiber is made from a polymethyl methacrylate core with a transparent polymer cladding. The optical fiber can have a diameter between approximately 0.75 mm and approximately 2.0 mm. In some embodiments, the optical fiber can have a diameter of about 0.75 mm, about 1 mm, about 1.5 mm, about 2 mm, less than about 0.75 mm or greater than about 2 mm as not all embodiments of the present disclosure are intended to be limited in this respect. In an embodiment, the optical fiber is made from a polymethyl methacrylate core with a transparent polymer cladding. It should be appreciated that the above-described characteristics and properties of the optical fibers are exemplary and not all embodiments of the present disclosure are intended to be limited in these respects. Light energy from a visible emitting light source can be transmitted by the optical fiber. In an embodiment, visible light having a wavelength spectrum of between about 380 nm to about 780 nm, between about 400 nm to about 600 nm, between about 420 nm to about 500 nm, between about 430 nm to about 440 nm, is used to cure the liquid light-curable material.

The presently disclosed embodiments provide expandable spinal devices of photodynamic bone stabilization systems of the present disclosure. It should be understood that any of the expandable spinal devices disclosed herein may include one or more radiopaque markers or bands, or may be fabricated from a material that is made to be radiopaque. For example, a radiopaque ink bead may be placed at a distal end of an expandable spinal device for alignment of the system during fluoroscopy. The one or more radiopaque bands and radiopaque ink bead, using radiopaque materials such as barium sulfate, tantalum, or other materials known to increase radiopacity, allows a medical professional to view the expandable spinal device during positioning to properly position the expandable spinal device during a repair procedure, and allows the medical professional to view the expandable spinal device during inflation and/or deflation. In an embodiment, the one or more radiopaque bands permit visualization of any voids that may be created by air that gets entrapped in the expandable spinal device.

It should be understood that any of the expandable spinal devices disclosed herein may be round, flat, cylindrical, oval, rectangular or any desired shape for a given application. The expandable spinal devices may be formed of a pliable, resilient, conformable, and strong material, including but not limited to urethane, polyethylene terephthalate (PET), nylon elastomer and other similar polymers. In an embodiment, an expandable spinal device of the present disclosure is constructed out of a PET nylon aramet or other non-consumable materials. In an embodiment, an expandable spinal device of the present disclosure may be formed from a material that allows the spinal device to conform to obstructions or curves at the site of implantation. In an embodiment, an expandable spinal device of the present disclosure may be formed from a material that includes or is made from natural or synthetic fibers, including, but not limited to, nylon fibers, polyester (PET) fibers, Polyethylene naphthalate (PEN) fibers, aramid fibers, ultra high molecular weight polyethylene (UHMWPE) fibers, polyethylene fibers, Poly (p-phenylene-2, 6-benzobisoxazole) (PBO) fibers, and carbon fibers.

It should be understood that any of the expandable spinal devices disclosed herein includes an outer surface that, in an embodiment, may be coated with materials such as, for example, drugs, bone glue, proteins, growth factors, or other coatings. For example, after a minimally invasive surgical procedure an infection may develop in a patient, requiring the patient to undergo antibiotic treatment. An antibiotic drug may be added to the outer surface of the expandable spinal device to prevent or combat a possible infection. Proteins, such as, for example, bone morphogenic protein or other growth factors have been shown to induce the formation of cartilage and bone. A growth factor may be added to the outer surface of the spinal device to help induce the formation of new bone. Due to the lack of thermal egress of the light-sensitive liquid in the spinal device, the effectiveness and stability of the coating is maintained.

It should be understood that the outer surface of the expandable spinal devices disclosed herein are resilient and puncture resistant. In an embodiment, the outer surface of the expandable spinal device is substantially even and smooth. In an embodiment, the outer surface of the expandable spinal device is not entirely smooth and may have some small bumps or convexity/concavity along the length. In an embodiment, the outer surface of the expandable spinal device may have ribs, ridges, bumps or other shapes to help the spinal device conform to the shape of the vertebrae or pedicles. In an embodiment, the expandable spinal device has a textured surface which provides one or more ridges that allow grabbing. In an embodiment, abrasively treating the outer surface of the expandable spinal device via chemical etching or air propelled abrasive media improves the connection and adhesion between the outer surface of the expandable spinal device and the surfaces of the vertebral body or pedicles. The surfacing significantly increases the amount of surface area that comes in contact with the bone resulting in a stronger grip.

The expandable spinal devices disclosed herein typically do not have any valves. One benefit of having no valves is that the expandable spinal device may be inflated or deflated as much as necessary to assist in the placement of the spinal device. Another benefit of the expandable spinal device having no valves is the efficacy and safety of the system. Since there is no communication passage of light-sensitive liquid to the body there cannot be any leakage of liquid because all the liquid is contained within the expandable spinal device. In an embodiment, a permanent seal is created between the expandable spinal device that is both hardened and affixed prior to the insertion catheter 101 being removed. The expandable spinal device may have valves, as all of the embodiments are not intended to be limited in this manner.

Intervertebral discs provide mobility and a cushion between the vertebrae. Degeneration of the intervertebral disc, often called “degenerative disc disease” (DDD) of the spine, is a condition that can be painful and can greatly affect the quality of one's life. While disc degeneration is a normal part of aging and for most people is not a problem, for certain individuals a degenerated disc can cause severe constant chronic pain. DDD may result from osteoarthritis, a herniated disc or spinal stenosis. In an embodiment, a photodynamic bone stabilization system of the present disclosure is used for treating degenerative disc disease. In an embodiment, the degenerative disc disease results from osteoarthritis. In an embodiment, the degenerative disc disease results from a herniated disc. In an embodiment, the degenerative disc disease results from spinal stenosis. In an embodiment, a photodynamic bone stabilization system of the present disclosure is used during an intervertebral disc arthroplasty procedure. In an embodiment, the photodynamic bone stabilization system includes a catheter having an elongated shaft with a proximal end adapter, a distal end releasably engaging an expandable interbody device, and a longitudinal axis therebetween; a light-conducting fiber configured to transmit light energy; and a liquid light-curable material. As described above with reference to FIG. 1, the catheter comprises an inner void sufficiently designed for passage of the liquid light-curable material to the expandable interbody device, and an inner lumen sufficiently designed for passage of the light-conducting fiber to the expandable interbody device. During use, the liquid light-curable material is delivered to the expandable interbody device to selectively expand the device and restore disc height. The liquid light-curable material remains within the expandable interbody device. When the device is expanded to a desired position, the light-conducting fiber is delivered to the expanded interbody device to transmit light energy to activate the initiator of the liquid light-curable material, which initiates polymerization of the liquid light-curable material and hardening of the interbody device in situ. In an embodiment, the interbody device of the present disclosure is sufficiently designed to restore spinal stability. In an embodiment, the interbody device of the present disclosure is sufficiently designed to restore nearly-normal physiologic mobility of spine. In an embodiment, the interbody device of the present disclosure is sufficiently designed to restore disc space height. In an embodiment, the interbody device of the present disclosure is sufficiently designed to restore an original disc height between an upper vertebral body and a lower vertebral body. In an embodiment, the interbody device of the present disclosure is sufficiently designed to restore an original disc height at an anterior portion, a middle portion and a posterior portion between an upper vertebral body and a lower vertebral body.

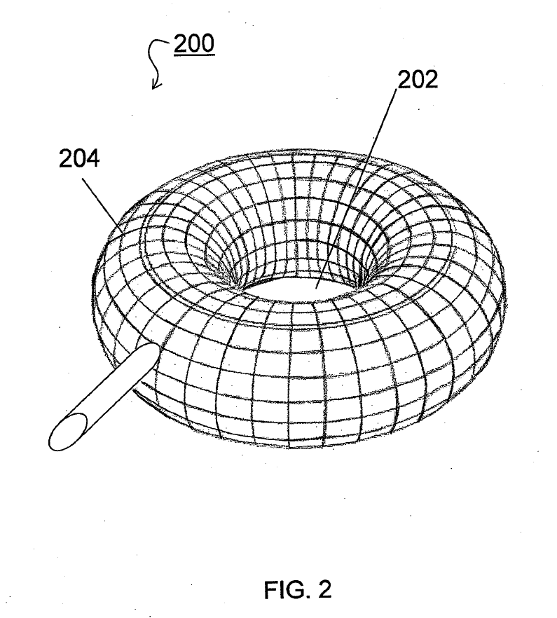

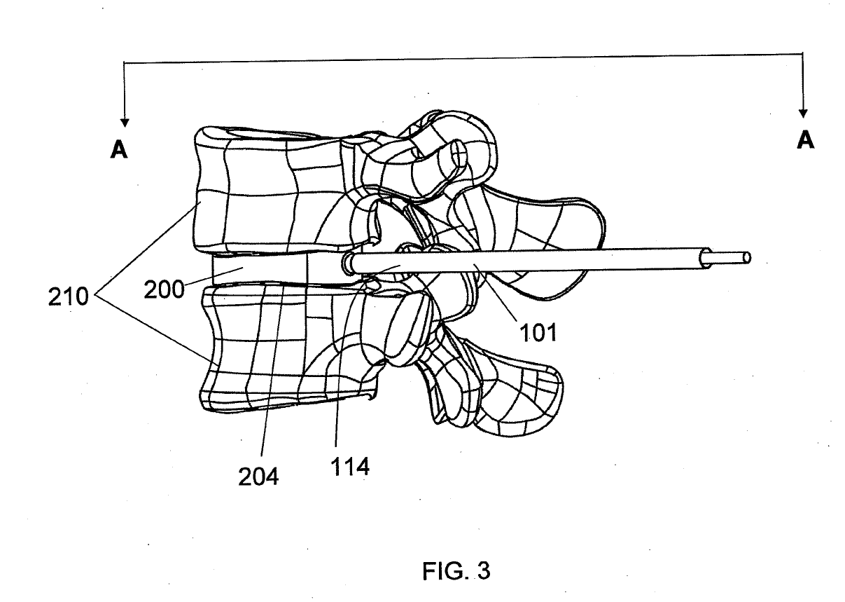

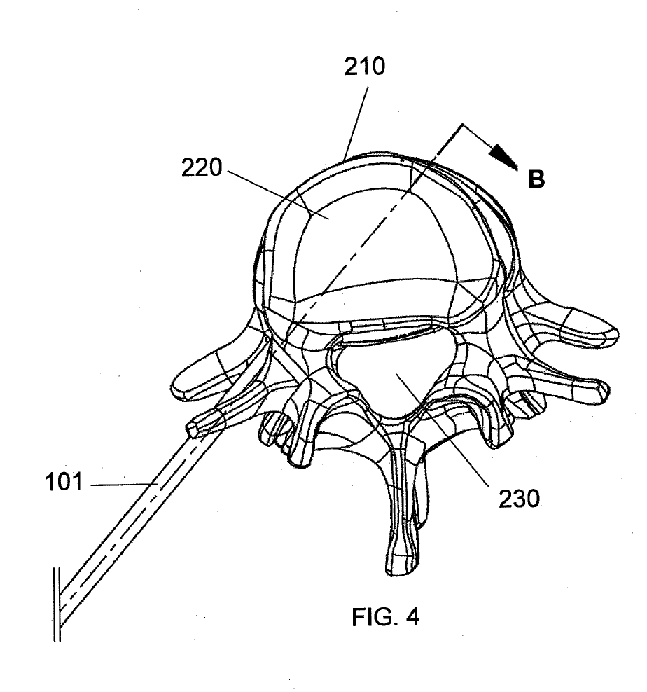

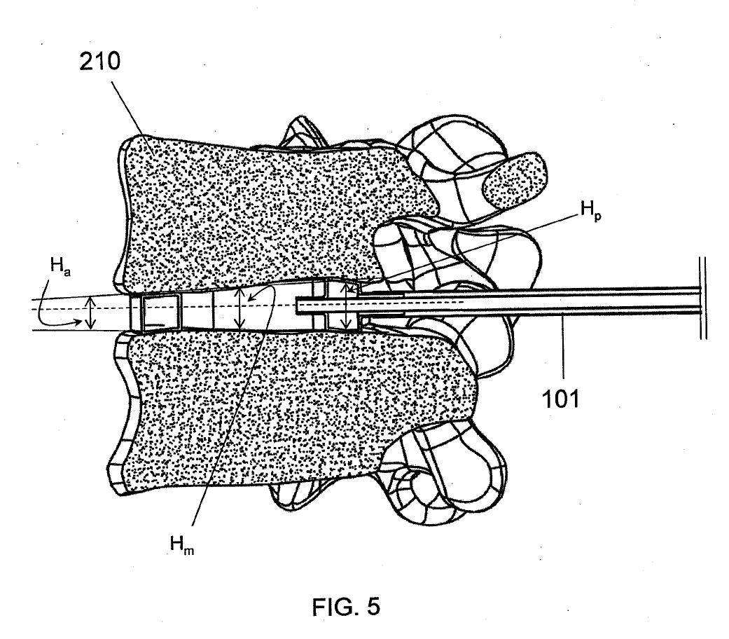

FIG. 2 in conjunction with FIG. 3, FIG. 4 and FIG. 5, show an embodiment of an interbody device 200 of the present disclosure positioned between two vertebrae 210 of a spinal column for the treatment of DDD. The interbody device 200 can help restore disc space height. In the embodiment depicted in FIG. 2, the interbody device 200 has a geometrical shape of a torus when fully inflated and cured with light-sensitive liquid. In an embodiment, the torus interbody device 200 has an interior space (hole) 202 in the middle and resembles, for example, a ring doughnut, a hula hoop or an inflated tire. It should be understood that in some embodiments, an interbody device of the present disclosure does not include an interior space, and instead may represent a filled doughnut. Outer surfaces 204 of the interbody device 200 provide support to two vertebrae 210 (illustrated in FIG. 3), and help restore disc height, while the interior space 202 can be filled with a bone graft or a bone graft substitute material possessing characteristics necessary for new bone growth—namely, osteoconductivity, osteogenicity, and osteoinductivity, thus allowing the two vertebrae to be fused together. The bone graft or bone graft substitute material supports the attachment of new osteoblasts and osteoprogenitor cells, providing an interconnected structure through which new cells can migrate and new vessels can form. Although the embodiment depicted in FIG. 2, FIG. 3, FIG. 4 and FIG. 5 show the interbody device 200 shaped as a torus, it should be understood that the interbody device 200 can have other shapes and still be within the scope and spirit of the presently disclosed embodiments. In an embodiment, a torus shaped interbody device is desirable, especially for fusion-type applications. The interbody device 200 may be rolled up or have creases and folds to accommodate insertion between vertebrae in a deflated state. In an embodiment, the interbody device 200 has a baffle structure which reduces wave motion of the light-sensitive liquid in the interbody device 200. Baffles would float within the interbody device 200 and may have serpentine, cone, coil or cylindrical shapes. The interbody device 200 may be a pad that is round, flat, cylindrical, oval, rectangular or another shape, as long as the interbody device 200 functions to restore disc height, improves spine function, and helps to eliminate debilitating pain. In an embodiment, the interbody device 200 has a first surface, an opposing second surface, and one or more side surfaces, for instance, cylinder-like. In an embodiment, bone graft can be placed around the interbody device 200.

In an embodiment, the interbody device 200 of the present disclosure, when implanted and inflated between two vertebral bodies, restores the posterior and anterior disc height. In an embodiment, the interbody device 200 of the present disclosure, when implanted and inflated between two vertebral bodies, restores the sagittal dimension and the coronal dimension of the damaged intervertebral disc. FIG. 3 shows the interbody device 200 during inflation. The interbody device 200 releasably engages a distal end 114 of the flexible insertion catheter 101 of FIG. 1. Light-sensitive liquid is introduced into the proximal end 112 of the insertion catheter 101 through port 135 and passes within the inner void of the insertion catheter 101 up into the interbody device 200 to move the interbody device 200 from a deflated state to an inflated state in situ. In an embodiment, the interbody device 200 has a pre-defined shape to fit between the two vertebrae 210, in the disc space, in an inflated state. However, since the user of the insertion catheter 101 has control over the delivery of the light-sensitive liquid to the interbody device 200, the interbody device 200 can be expanded such that at least a portion of the outer surface 204 of the interbody device 200 contacts the upper and lower vertebrae 210. In an embodiment, the interbody device 200 can be expanded such that at least a portion of the outer surface 204 of the interbody device 200 contacts the upper and lower vertebrae 210 at all locations along the surfaces of the vertebrae 210.

In the top-down plan view of FIG. 4, it can be seen that the interbody device 200 may not significantly extend beyond the body 220 of the vertebrae 210, nor impinge upon the spinal canal 230. In an embodiment, the interbody device 200 is delivered to the spine by the flexible insertion catheter 101 from the posterior aspect of a patient, as illustrated in FIG. 5. The posterior approach taken to place the interbody device 200 with a small delivery profile is advantageous over the anterior approach, which is typically required to place a large implant. Having the ability to insert a large interbody device with a posterior technique has significant benefits. First, the typical ALIF (anterior lumbar interbody fusion) device is larger and offers better support than the standard PLIF (posterior lumbar interbody fusion) device. This larger ALIF type implant contacts the stronger outer portion of the vertebral body leading to a better procedure. An anterior procedure is needed to place the larger implant as the posterior structures do not allow adequate access. There are other conditions, such as obesity that make an anterior approach very difficult or previous abdominal surgery that make the anterior approach very risky. For these reasons, an expanding interbody device 200 of the present disclosure that can navigate through the posterior structures and be placed by a posterior technique is beneficial.

The interbody device 200 and method of delivering the interbody device 200 may provide custom matched geometry to every patient with substantial or near total contact between the outer surface 204 of the inflated interbody device 200 and the vertebrae 210, as further illustrated in FIG. 5. The minimally invasive surgical method used to deliver the interbody device 200 via the flexible insertion catheter 101 percutaneously may reduce the chances of damaging the surrounding tissue during insertion. In an embodiment of a method disclosed herein, there may be no need to remove facets from the vertebrae.

Although FIG. 3 and FIG. 5 illustrate the top vertebrae 210 and the bottom vertebrae 210 approximately parallel to one another, depending on where in the spinal column the DDD occurs, the top vertebrae 210 and the bottom vertebrae 210 may be mis-aligned. The anterior (Hₐ), middle (Hₘ), and posterior (Hₚ) disc height (see FIG. 5) may vary depending on where the DDD is within the spinal column. Therefore, an advantage of the interbody device 200 of the present disclosure is the ability for a user to deliver the appropriate amount of light-sensitive liquid to the interbody device 200, and subsequently cure the liquid, to create an interbody device 200 that substantially conforms to the surrounding environment.

In an embodiment, the thickness of the inflated interbody device 200 varies in different positions within the intervertebral disc portion. For example, the anterior portion of the interbody device 200 can have thickness of about 8-14 mm, the middle portion of the interbody device 200 can have a thickness of about 6-14 mm, and the posterior portion of the interbody device 200 can have a thickness of about 3-12 mm depending on the level. The interbody device 200 can have an elliptical shape having a anterior-posterior dimension of 20-50 mm and a medio-lateral dimension of 30-70 mm Those skilled in the art will recognize that variations within these ranges are possible and still within the scope and spirit of the presently disclosed embodiments.

A method is provided for treatment of degenerative disc disease using a photodynamic bone stabilization system of the present disclosure. In an embodiment, the photodynamic bone stabilization system includes a thin-walled, non-compliant, expandable interbody device releasably mounted on a small diameter, flexible insertion catheter. In an embodiment, the expandable interbody device has an interior space (hole) in the middle and resembles a ring doughnut, a hula hoop or an inflated tire. A minimally invasive incision is made through a skin of a patient, i.e. percutaneously. In an embodiment, a posterior approach is taken to reach the spine. An introducer sheath may be introduced to reach the spine. In an embodiment, at least a portion of a damaged intervertebral disc between an upper vertebral body and a lower vertebral body is removed. The interbody device is delivered to the intervertebral space in a deflated state as it is steered into position by the flexible insertion catheter under fluoroscopy. In an embodiment, the interbody device replaces the central portion of the disc (Nucleus Pulposus). In an embodiment, the interbody device replaces the whole disc including the Disc Wall (Annulus). The location of the device member may be determined using at least one radiopaque marker which is detectable from outside or inside the intervertebral space. The interbody device is placed in the intervertebral space. Once the interbody device is in the correct position between the two vertebrae, the introducer sheath may be removed. A delivery system housing a light-sensitive liquid is attached to the proximal end of the insertion catheter. The light-sensitive liquid is then infused through an inner void in the insertion catheter and enters the interbody device. This addition of the light-sensitive liquid within the interbody device causes the interbody device to expand. As the interbody device is expanded, the intervertebral disc height is restored.

Once the orientation of the interbody device is confirmed to be in a desired position, the liquid light-curable material may be cured within the interbody device, such as by illumination with a visible emitting light-conducting fiber that is placed within the inner lumen of the insertion catheter up into the interbody device. In an embodiment, visible light having a wavelength spectrum of between about 380 nm to about 780 nm, between about 400 nm to about 600 nm, between about 420 nm to about 500 nm, between about 430 nm to about 440 nm, is used to cure the liquid light-curable material. In an embodiment, the addition of the light causes the photoinitiator in the liquid light-curable material, to initiate the polymerization process: monomers and oligomers join together to form a durable biocompatible crosslinked polymer. In an embodiment, the cure provides complete 360 degree radial and longitudinal support and stabilization to the intervertebral space. In an embodiment, during the curing phase, a syringe housing cooling medium is attached to the proximal end of the insertion catheter and continuously delivered to the interbody device via the inner lumen to control polymerization temperature. In an embodiment, the cooling medium can be collected by connecting tubing to the distal end of the inner lumen and collecting the cooling medium. In an embodiment, the cooling medium can be maintained in the interior space of the interbody device. In an embodiment, during the curing phase, a syringe housing pressurizing medium is attached to the proximal end of the insertion catheter and continuously delivered to the interbody device via the inner lumen to control polymerization shrinkage. After the liquid light-curable material has been hardened, the light-conducting fiber can be removed from the insertion catheter. The interbody device once hardened, may be released from the insertion catheter. The hardened interbody device remains in the intervertebral space, and the insertion catheter is removed. The outer surface of the hardened interbody device makes contact with the bodies of the vertebrae, either partially or totally. Once the cured interbody device is in place, bone graft or bone graft substitute material may be inserted into the interior space or around the hardened expandable interbody device. In an embodiment, the bone graft substitute material can be inserted into the interior space using the same inner lumen that previously housed the light-conducting fiber. The bone graft substitute material creates fusion between the two vertebral bodies. In an embodiment, the interbody device can replicate the complex movement patterns of a natural disc.

In spinal fusion (arthrodesis), two or more vertebrae are permanently healed or fused together. Arthrodesis refers to the entire spectrum of stabilization including flexible, as well as rigid procedures. Fusion eliminates motion between vertebrae and prevents the slippage from worsening after surgery. Spinal fusion surgery is an aggressive surgery, and current techniques require muscle splitting, an invasive technique that can require extended rehabilitation. Spinal fusion surgery may be required for patients having any one of the following conditions, including, but not limited to, degenerative disc disease, spinal disc herniation, discogenic pain, spinal tumor, spinal stenosis, vertebral fracture, scoliosis, kyphosis, spondylolisthesis, spondylosis, Posterior Rami Syndrome, other degenerative spinal conditions and any condition that causes instability of the spine.

In an embodiment, a photodynamic bone stabilization system of the present disclosure is used during a spinal fusion surgery. In an embodiment, a photodynamic bone stabilization system of the present disclosure is used during a stabilization surgery. In an embodiment, the photodynamic bone stabilization system includes a catheter having an elongated shaft with a proximal end adapter, a distal end releasably engaging an expandable spinal fusion device, and a longitudinal axis therebetween; a light-conducting fiber configured to transmit light energy; and a liquid light-curable material. As described above with reference to FIG. 1, the catheter comprises an inner void sufficiently designed for passage of the liquid light-curable material to the expandable spinal fusion device, and an inner lumen sufficiently designed for passage of the light-conducting fiber to the expandable spinal fusion device. During use, the liquid light-curable material is delivered to the expandable spinal fusion device to selectively expand the device and fixate the spinal segment. The liquid light-curable material remains within the expandable spinal fusion device. When the device is expanded to a desired position, the light-conducting fiber is delivered to the expanded spinal fusion device to transmit light energy to activate the initiator of the liquid light-curable material, which initiates polymerization of the liquid light-curable material and hardening of the spinal fusion device in situ. In an embodiment, the thickness of the expanded spinal fusion device is about 5 mm. An inflated spinal fusion device may have a size ranging from about 5 mm to about 7 mm by about 25 mm to about 150 mm. Those skilled in the art will recognize that variations within these ranges are possible and still within the scope and spirit of the presently disclosed embodiments.

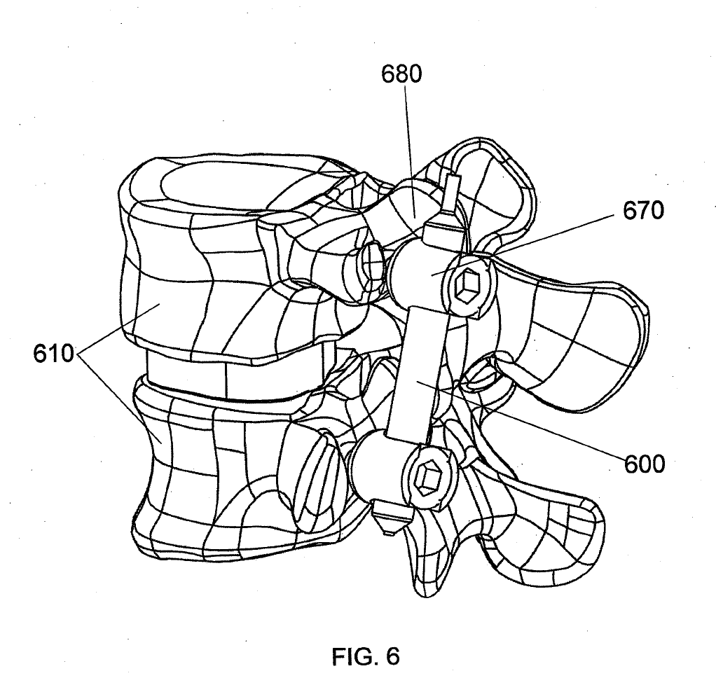

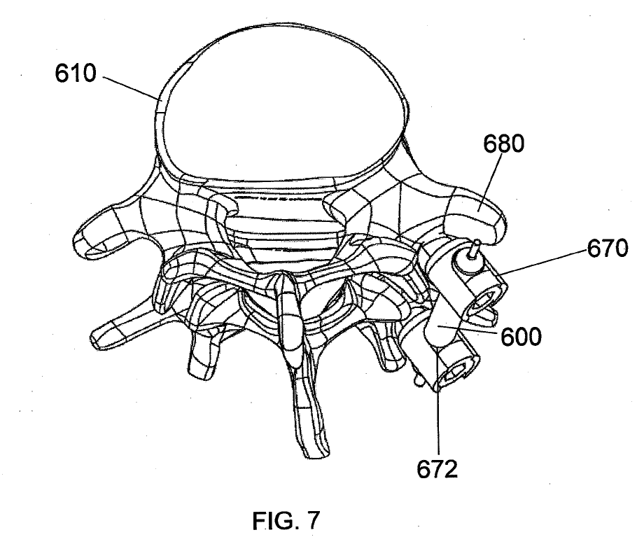

FIG. 6 and FIG. 7 show embodiments of a spinal fusion device 600 of the present disclosure for providing fusion and stabilization of a spinal segment. FIG. 6 shows pedicle screws 670 affixed to the pedicles 680 of the vertebrae 610. As known in the art, pedicle screws 670 provide a means of gripping a spinal segment, typically on the pedicles 680 (radices arci vertebrae). The pedicle screws 670 themselves do not fixate the spinal segment, but act as firm anchor points for attachment to the spinal fusion device 600 of the present disclosure. Pedicle screws 670 have openings 672 to allow the deflated spinal fusion device 600 to pass therethrough (FIG. 7—which shows a cured spinal fusion device 600). In an embodiment, the spinal fusion device 600, shown in an inflated state, fully engages the pedicle screws 670. In an embodiment, pedicle screws 670 may be placed at two consecutive spine segments (for example, at lumbar segment 4 and 5), and connected by spinal fusion device 600 to prevent or reduce motion between spinal segments. In an alternative embodiment, pedicle screws 670 may be placed at three consecutive spine segments and connected by spinal fusion device 600. An advantage of the expandable spinal fusion device 600 is that the spinal fusion device 600 may provide custom matched rod geometry to every patient, on a patient-by-patient basis. There is no need to bend or contour the spinal fusion device 600. In an embodiment, the spinal fusion device 600 may adapt to mixed and different types of pedicle screws 670 in the same patient, as long as the pedicle screws 670 have openings therethrough through which the spinal fusion device 600 may be inserted.

In a typical procedure for spinal fusion, the dorsal muscles need to be split or dissected to gain access to the vertebrae. An advantage of the spinal fusion device 600 of the present disclosure is that this step is not required, because the small delivery profile of the spinal fusion device 600, in a deflated state, allows for a minimally invasive rod insertion. The outcome after surgery is greatly influenced by the condition of surrounding soft tissues.

A method is provided for spinal fusion using a photodynamic bone stabilization system of the present disclosure. In an embodiment, the photodynamic bone stabilization system includes a flexible catheter having an elongated shaft with a proximal end adapter, a distal end releasably engaging an expandable spinal fusion device, and a longitudinal axis therebetween. A minimally invasive incision is made through a skin of a patient, i.e. percutaneously. In an embodiment, a posterior approach is taken to reach the spine. Pedicle screws are placed at the appropriate locations, usually two or three consecutive spine segments. An introducer sheath may be introduced to reach the spine. The spinal fusion device is positioned into the openings of the affixed pedicle screws. The spinal fusion device is delivered in a deflated state as the device is steered into position by the flexible insertion catheter under fluoroscopy. The location of the spinal fusion device may be determined using at least one radiopaque marker which is detectable. The spinal fusion device is inserted through the holes in the pedicle screws in a deflated state. Once the spinal fusion device is in the correct position, the introducer sheath may be removed. A delivery system housing the liquid light-curable material is attached to the proximal end adapter of the insertion catheter. The liquid light-curable material is then infused through an inner void in the insertion catheter and enters the spinal fusion device. This addition of the liquid light-curable material within the spinal fusion device causes the spinal fusion device to expand. As the spinal fusion device is expanded, the pedicle screws and associated vertebrae become a more rigid unit.

Once the orientation of the spinal fusion device is confirmed to be in a desired position, the liquid light-curable material may be cured within the spinal fusion device (in situ), such as by illumination with a visible emitting light-conducting fiber that is placed within the inner lumen of the insertion catheter up into the spinal fusion device. In an embodiment, visible light having a wavelength spectrum of between about 380 nm to about 780 nm, between about 400 nm to about 600 nm, between about 420 nm to about 500 nm, between about 430 nm to about 440 nm, is used to cure the liquid light-curable material. In an embodiment, the addition of the light causes the photoinitiator in the liquid light-curable material, to initiate the polymerization process: monomers and oligomers join together to form a durable biocompatible crosslinked polymer. In an embodiment, the cure provides complete 360 degree radial and longitudinal support and stabilization to the pedicle screws and associated vertebrae. In an embodiment, during the curing phase, a syringe housing cooling medium is attached to the proximal end of the insertion catheter and delivered to the spinal fusion device to control polymerization temperature. In an embodiment, the cooling medium can be maintained in the interior space of the spinal fusion device. In an embodiment, during the curing phase, a syringe housing pressurizing medium is attached to the proximal end of the insertion catheter and continuously delivered to the spinal fusion device via the inner lumen to control polymerization shrinkage. After the liquid light-curable material has been hardened, the light-conducting fiber can be removed from the insertion catheter. The spinal fusion device once hardened, may be released from the insertion catheter. The hardened spinal fusion device remains engaged to the pedicle screws, and the insertion catheter is removed. A final tightening of the pedicle screws can complete the assembly. Optionally, once the cured spinal fusion device is in place, bone graft or bone graft substitute material can be inserted near the hardened expandable spinal fusion device. In an embodiment, the bone graft substitute material can be inserted near the hardened expandable spinal fusion device using the same inner lumen that previously housed the light-conducting fiber. In an embodiment, the bone graft substitute material helps create fusion between the vertebral bodies.

In an embodiment, a photodynamic bone stabilization system includes a catheter having an elongated shaft with a proximal end adapter, a distal end releasably engaging an expandable interspinous process spacer device, and a longitudinal axis therebetween; a light-conducting fiber configured to transmit light energy; and a liquid light-curable material. As described above with reference to FIG. 1, the catheter comprises an inner void sufficiently designed for passage of the liquid light-curable material into the expandable interspinous process spacer device, and an inner lumen sufficiently designed for passage of the light-conducting fiber into the expandable interspinous process spacer. During use, the liquid light-curable material is delivered to the expandable interspinous process spacer device to selectively expand the device and fixate the spinal segment. The liquid light-curable material remains within the expandable interspinous process spacer device. When the device is expanded to a desired position, the light-conducting fiber is delivered to the expanded interspinous process spacer device to transmit light energy to activate the initiator of the liquid light-curable material, which initiates polymerization of the liquid light-curable material and hardening of the interspinous process spacer device in situ. In an embodiment, the interspinous process spacer device of the present disclosure is sufficiently designed to distract (open) the foramen, where the nerve endings pass away from the center of the spinal region and into the legs. In an embodiment, the interspinous process spacer device of the present disclosure is sufficiently designed to unload the intervertebral disc. In an embodiment, the interspinous process spacer device of the present disclosure is sufficiently shaped to allow controlled movement in forward and backward bending. In an embodiment, the interspinous process spacer device of the present disclosure is sufficiently designed to restrict painful motion while enabling otherwise normal motion.

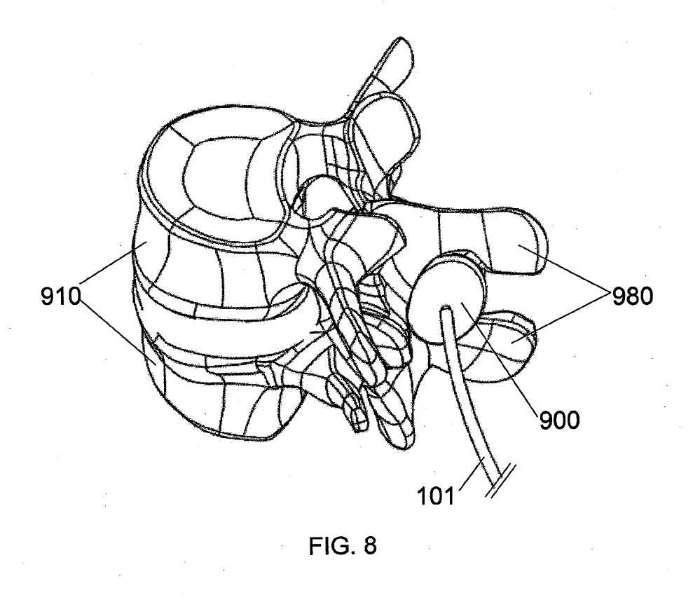

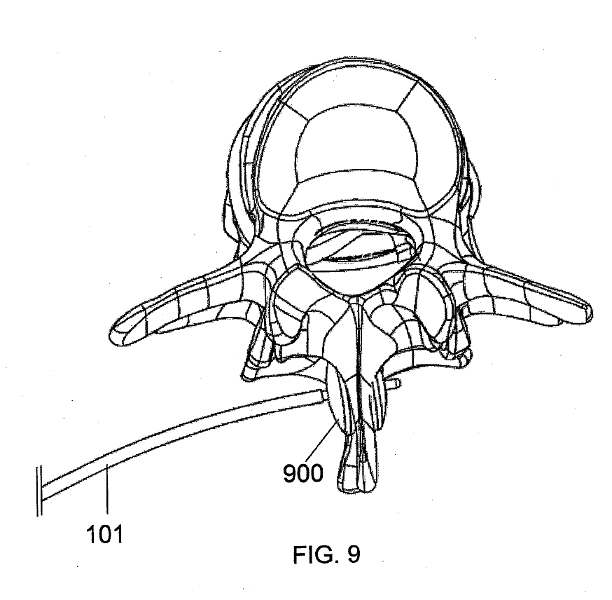

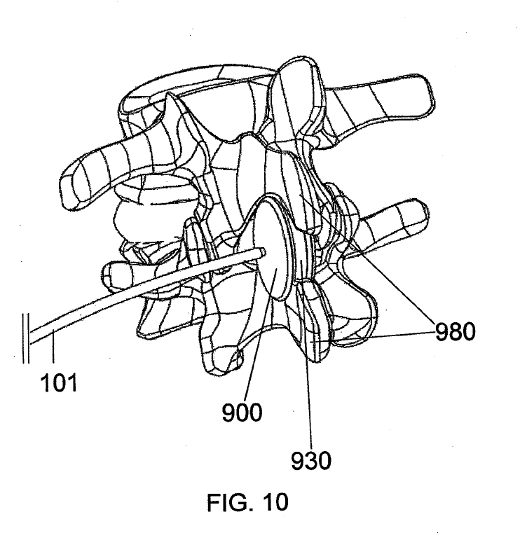

FIG. 8, FIG. 9 and FIG. 10 show an embodiment of a interspinous process spacer device 900 positioned between two spinous processes 980 of a spinal column for providing dynamic stabilization (also known as soft stabilization or flexible stabilization) of a spinal segment. In an embodiment, the interspinous process spacer device 900 is formed from a pliable, resilient, conformable, and strong material, and includes a circumferential groove 930. The interspinous process spacer device 900 is sufficiently designed to inflate and deflate as a liquid light-curable material is added to the device 900. The device 900 when positioned between the spinous processes 980 is inflated such that the groove 930 engages the upper spinous process and the lower spinous process to restrict painful motion while enabling otherwise normal motion. In FIG. 8 there can be seen the flexible insertion catheter 101 and the interspinous process spacer device 900 shown in an inflated state after liquid light-curable material is added to the interspinous process spacer device 900. Interspinous process spacer device 900 is placed between two spinous processes 980 of adjacent vertebrae. In an embodiment, interspinous process spacer device 900 is sufficiently designed to alter the load bearing pattern of the motion segment and to control any abnormal motion while leaving the spinal segment mobile.

In an embodiment, the interspinous process spacer device 900 may have a pre-defined shape to engage the spinous processes 980. In an embodiment, the interspinous process spacer device 900 is shaped as a pad that is round, flat, cylindrical, oval, rectangular or another shape, the pad having a groove 930 for engaging the spinous processes. In an embodiment, the interspinous process spacer device 900 has a first surface, an opposing second surface, and one or more side surfaces, for instance, puck-like or cylinder-like. For example, as depicted in the embodiments of FIG. 8, FIG. 9 and FIG. 10, the pre-defined shape of the interspinous process spacer device 900 substantially resembles a grooved hockey puck or pulley wheel. As illustrated in FIG. 10, the interspinous process spacer 900 has groove 930 into which the spinous processes 980 are accommodated. In an embodiment, the interspinous process spacer device 900 may be delivered to the spine by the flexible insertion catheter 101 from the posterior aspect of a patient, as seen in FIG. 9. The posterior approach taken to place the interspinous process spacer device 900 with a small delivery profile is advantageous over the anterior approach, which is typically required to place a large implant.

The interspinous process spacer 900, in a deflated state, may be inserted through only a small incision (3-4 mm). A small incision made for delivering the interspinous process spacer device 900 may minimize the risk of wound dehiscence, have a low rate of surgical complications, and promote rapid recovery.

In an embodiment, the interspinous process spacer device 900 is self-dilating, that is, the interspinous process spacer device 900 may separate tissues during inflation. In an embodiment, the interspinous process spacer device 900 is self-distracting, that is, the interspinous process spacer device 900 may separate the spinous processes of adjacent vertebrae during inflation. According to the present disclosure, the interspinous process spacer device 900 can be used with a variety of durometer liquid, light-curable materials for the desired support, such as for fusion or for dynamic support of the spinous processes 980. The interspinous process spacer device 900 may limit pathological extension of the spine. The interspinous process spacer device 900 may preserve mobility and anatomical structures.

An embodiment of a method for treatment of spinal stenosis using a photodynamic bone stabilization system of the present disclosure is disclosed herein. The photodynamic bone stabilization system includes a flexible catheter having an elongated shaft with a proximal end adapter, a distal end releasably engaging an expandable interspinous process spacer device, and a longitudinal axis therebetween. A minimally invasive incision is made through a skin of the patient, i.e. percutaneously. In an embodiment, a posterior approach is taken to reach the spine and spinal processes. An introducer sheath may be introduced to reach the spine. The interspinous process spacer device is delivered to the processes in a deflated state as it is steered into position by the flexible insertion catheter under fluoroscopy. The location of the interspinous process spacer device may be determined using at least one radiopaque marker which is detectable. The interspinous process spacer device is placed between two spinous processes. Once the interspinous process spacer device is in the correct position between the two spinous processes, the introducer sheath may be removed. A delivery system housing a light-sensitive liquid monomer is attached to the proximal end adapter of the insertion catheter. The light-sensitive liquid monomer is then infused through an inner void in the insertion catheter and enters the interspinous process spacer device. This addition of the light-sensitive liquid monomer within the interspinous process spacer device causes the interspinous process spacer device to expand. As the interspinous process spacer device is expanded, spinal processes are supported, and spinal stenosis may be alleviated.

Once the orientation of the interspinous process spacer device is confirmed to be in a desired position, the liquid light-curable material may be cured within the spinous process device, such as by illumination with a visible emitting light source. In an embodiment, visible light having a wavelength spectrum of between about 380 nm to about 780 nm, between about 400 nm to about 600 nm, between about 420 nm to about 500 nm, between about 430 nm to about 440 nm, is used to cure the liquid light-curable material. In an embodiment, the addition of the light causes the photoinitiator in the liquid light-curable material, to initiate the polymerization process: monomers and oligomers join together to form a durable biocompatible crosslinked polymer. In an embodiment, the cure provides complete 360 degree radial and longitudinal support and stabilization to the spinous processes. In an embodiment, during the curing phase, a syringe housing cooling medium is attached to the proximal end of the insertion catheter and delivered to the spinal fusion device to control polymerization temperature. In an embodiment, the cooling medium can be collected by connecting tubing to the distal end of the inner lumen and collecting the cooling medium. In an embodiment, the cooling medium can be maintained in the interior space of the interspinous process spacer device. In an embodiment, during the curing phase, a syringe housing pressurizing medium is attached to the proximal end of the insertion catheter and continuously delivered to the interspinous process spacer device via the inner lumen to control polymerization shrinkage. After the liquid light-curable material has been hardened, the light-conducting fiber can be removed from the insertion catheter. The interspinous process spacer device once hardened, may be released from the insertion catheter. The hardened interspinous process spacer device remains spanning the two processes, and the insertion catheter is removed.

An interspinous process spacer system includes a light-conducting fiber configured to transmit light energy; a liquid light-curable material; and a catheter having an elongated shaft with a proximal end adapter, a distal end releasably engaging an expandable interspinous process spacer device, and a longitudinal axis therebetween, wherein an inner void of the catheter is sufficiently designed for passage of the liquid light-curable material to the expandable interspinous process spacer device, wherein an inner lumen of the catheter is sufficiently designed for passage of the light-conducting fiber to the expandable interspinous process spacer device, wherein the expandable interspinous process spacer device includes a circumferential groove, wherein the expandable interspinous process spacer device is sufficiently designed to inflate and deflate as the liquid light-curable material is added, and wherein the expandable interspinous process spacer device, when positioned between two spinous processes and inflated, is configured to engage the spinous processes at the groove.

A method includes providing a system comprising: a light-conducting fiber configured to transmit light energy; a liquid light-curable material; and a catheter having an elongated shaft with a proximal end adapter, a distal end releasably engaging an expandable interbody device, and a longitudinal axis therebetween, wherein an inner void of the catheter is sufficiently designed for passage of the liquid light-curable material to the expandable interbody device, wherein an inner lumen of the catheter is sufficiently designed for passage of the light-conducting fiber to the expandable interbody device, and wherein the expandable interbody device is sufficiently designed to inflate and deflate as the liquid light-curable material is added; removing at least a portion of a damaged intervertebral disc, the damaged intervertebral disc positioned between an upper vertebral body and a lower vertebral body; inserting the expandable interbody device between the upper vertebral body and the lower vertebral body in place of the damaged intervertebral disc; infusing the liquid light-curable material into the expandable interbody device to inflate the expandable interbody device; inserting the light-conducting fiber into the inner lumen of the catheter so that the light-conducting fiber resides in the expandable interbody device; activating the light-conducting fiber to transmit light energy to the expandable interbody device to initiate in situ polymerization of the liquid light-curable material within the expandable interbody device; and completing the in situ polymerization of the liquid light-curable material to harden the expandable interbody device, wherein at least a portion of an outer surface of the hardened expandable interbody device engages the upper vertebral body and the lower vertebral body.

A method includes providing a system comprising: a light-conducting fiber configured to transmit light energy; a liquid light-curable material; and a catheter having an elongated shaft with a proximal end adapter, a distal end releasably engaging an expandable spinal fusion device, and a longitudinal axis therebetween, wherein an inner void of the catheter is sufficiently designed for passage of the liquid light-curable material to the expandable spinal fusion device, wherein an inner lumen of the catheter is sufficiently designed for passage of the light-conducting fiber to the expandable spinal fusion device, and wherein the expandable spinal fusion device is sufficiently designed to inflate and deflate as the liquid light-curable material is added; placing pedicle screws at consecutive spine segments, each of the pedicle screws having openings; inserting the expandable spinal fusion device into the openings of the pedicle screws to connect the pedicle screws together; infusing the liquid light-curable material into the expandable spinal fusion device to inflate the expandable spinal fusion device; inserting the light-conducting fiber into the inner lumen of the catheter so that the light-conducting fiber resides in the expandable spinal fusion device; activating the light-conducting fiber to transmit light energy to the expandable spinal fusion device to initiate in situ polymerization of the liquid light-curable material within the expandable spinal fusion device; and completing the in situ polymerization of the liquid light-curable material to harden the expandable spinal fusion device, wherein the hardened expandable spinal fusion device is sufficiently designed to fixate the spine segment.

All patents, patent applications, and published references cited herein are hereby incorporated by reference in their entirety. It will be appreciated that several of the above-disclosed and other features and functions, or alternatives thereof, may be desirably combined into many other different systems or application. Various presently unforeseen or unanticipated alternatives, modifications, variations, or improvements therein may be subsequently made by those skilled in the art.

What is claimed is:

1. An interspinous process spacer system comprising:
a light-conducting fiber configured to transmit light energy;
a liquid light-curable material; and
a catheter having an elongated shaft with a proximal end adapter, a distal end releasably engaging an expandable interspinous process spacer device, and a longitudinal axis therebetween,
wherein an inner void of the catheter is sufficiently designed for passage of the liquid light-curable material to the expandable interspinous process spacer device,
wherein an inner lumen of the catheter is sufficiently designed for passage of the light-conducting fiber to the expandable interspinous process spacer device,
wherein the expandable interspinous process spacer device includes a circumferential groove,
wherein the expandable interspinous process spacer device is sufficiently designed to inflate and deflate as the liquid light-curable material is added, and
wherein the expandable interspinous process spacer device, when positioned between two spinous processes and inflated, is configured to engage the spinous processes at the groove.

2. The system of claim 1 wherein the proximal end adapter comprises:
a first adapter for infusion of the liquid light-curable material; and
a second adapter for introduction of the light-conducting fiber.

3. The system of claim 1 wherein the light-conducting fiber is an optical fiber configured to transmit light with a wavelength between about 420 nanometers and about 500 nanometers.

4. The system of claim 1 wherein the expandable interspinous process spacer device is fabricated from a thin-walled, non-compliant PET nylon aramet.

5. The system of claim 1 wherein the expandable interspinous process spacer device is shaped to resemble a grooved pulley wheel.

6. The system of claim 1 for use in treating spinal stenosis.

7. A method comprising:
providing a system comprising:
a light-conducting fiber configured to transmit light energy;
a liquid light-curable material; and
a catheter having an elongated shaft with a proximal end adapter, a distal end releasably engaging an expandable interbody device, and a longitudinal axis therebetween,
wherein an inner void of the catheter is sufficiently designed for passage of the liquid light-curable material to the expandable interbody device,
wherein an inner lumen of the catheter is sufficiently designed for passage of the light-conducting fiber to the expandable interbody device, and
wherein the expandable interbody device is sufficiently designed to inflate and deflate as the liquid light-curable material is added;
removing at least a portion of a damaged intervertebral disc, the damaged intervertebral disc positioned between an upper vertebral body and a lower vertebral body;
inserting the expandable interbody device between the upper vertebral body and the lower vertebral body in place of the damaged intervertebral disc;
infusing the liquid light-curable material into the expandable interbody device to inflate the expandable interbody device;
inserting the light-conducting fiber into the inner lumen of the catheter so that the light-conducting fiber resides in the expandable interbody device;
activating the light-conducting fiber to transmit light energy to the expandable interbody device to initiate polymerization of the liquid light-curable material within the expandable interbody device; and
completing the polymerization of the liquid light-curable material to harden the expandable interbody device,
wherein at least a portion of an outer surface of the hardened expandable interbody device engages the upper vertebral body and the lower vertebral body.

8. The method of claim 7 wherein insertion of the expandable interbody device between the upper vertebral body and the lower vertebral body is by a posterior approach.

9. The method of claim 7 wherein the expandable interbody device is fabricated from a thin-walled, non-compliant PET nylon aramet.

10. The method of claim 7 wherein the hardened expandable interbody device restores an original disc height between the upper vertebral body and the lower vertebral body.

11. The method of claim 7 wherein the hardened expandable interbody device restores an original disc height at an anterior portion, a middle portion and a posterior portion between the upper vertebral body and the lower vertebral body.

12. The method of claim 7 wherein a thickness of the hardened expandable interbody device is constant as the hardened expandable interbody device engages the upper vertebral body and the lower vertebral body.

13. The method of claim 7 wherein a thickness of the hardened expandable interbody device varies as the hardened expandable interbody device engages the upper vertebral body and the lower vertebral body.

14. The method of claim 7 further comprising removing the light-conducting fiber from the catheter.

15. The method of claim 7 further comprising releasing the expandable interbody device from the catheter.

16. The method of claim 7 further comprising placing bone graft around the interbody device.

17. A method comprising:
providing a system comprising:
a light-conducting fiber configured to transmit light energy;
a liquid light-curable material; and
a catheter having an elongated shaft with a proximal end adapter, a distal end releasably engaging an expandable spinal fusion device, and a longitudinal axis therebetween,
wherein an inner void of the catheter is sufficiently designed for passage of the liquid light-curable material to the expandable spinal fusion device,
wherein an inner lumen of the catheter is sufficiently designed for passage of the light-conducting fiber to the expandable spinal fusion device, and
wherein the expandable spinal fusion device is sufficiently designed to inflate and deflate as the liquid light-curable material is added;
placing pedicle screws at consecutive spine segments, each of the pedicle screws having openings;
inserting the expandable spinal fusion device into the openings of the pedicle screws to connect the pedicle screws together;
infusing the liquid light-curable material into the expandable spinal fusion device to inflate the expandable spinal fusion device;
inserting the light-conducting fiber into the inner lumen of the catheter so that the light-conducting fiber resides in the expandable spinal fusion device;
activating the light-conducting fiber to transmit light energy to the expandable spinal fusion device to initiate polymerization of the liquid light-curable material within the expandable spinal fusion device; and
completing the polymerization of the liquid light-curable material to harden the expandable spinal fusion device,
wherein the hardened expandable spinal fusion device is sufficiently designed to fixate the spine segment.

18. The method of claim 17 further comprising removing the light-conducting fiber from the catheter.

19. The method of claim 17 further comprising releasing the expandable spinal fusion device from the catheter.

20. The method of claim 17 for use in treating a condition selected from the group consisting of degenerative disc disease, spinal disc herniation, discogenic pain, spinal tumor, spinal stenosis, vertebral fracture, scoliosis, kyphosis, spondylolisthesis, spondylosis, Posterior Rami Syndrome, other degenerative spinal conditions and any condition that causes instability of the spine.
